(19) United States
(12) Patent Application Publication
(10) Pub. No.: US 2007/0218024 A1
(43) Pub. Date: Sep. 20, 2007

(54) Dry water cosmetic compositions that change color upon application

(76) Inventors: Tatyana Zamyatin, Princeton Junction, NJ (US); Ida Marie Sandewicz, Monroe Township, NJ (US); Julio Gans Russ, Westfield, NJ (US); Sarah K. Jabush, Belford, NJ (US)

Correspondence Address: JULIE BLACKBURN;REVLON CONSUMER PRODUCTS CORPORATION, 237 PARK AVENUE, NEW YORK, NY, 10017, US

(21) Appl. No.: 11/378,827

(22) Filed: Mar. 17, 2006

(51) Int. Cl. A61K 8/73

(52) U.S. Cl. 424/63; 424/64; 424/70.13

(57) ABSTRACT

A powdered water cosmetic composition comprising activatable pigments, a water phase comprised of water droplets, and a particulate phase comprised of non-wetting particulates operable to surround and sequester the individual water droplets from each other in the composition, wherein said activatable pigments prior to activation provide a composition having a first resting color and, upon activation by application to a keratinous surface, a second application color which is visually distinct from the first color, and a method for applying color to keratinous surfaces using the composition of the invention.

### TECHNICAL FIELD

The invention is in the field of aqueous based color cosmetic compositions in the powdered water form that change color upon application to the desired keratinous surface.

### BACKGROUND OF INVENTION

The term “powdered water” or “dry water” refers to compositions that exist in the dry powder form yet contain appreciable amounts of water. Powdered water compositions are typically made by combining water or water soluble solvents with certain hydrophobic non-wetting particulate materials such as silica. When the water is mixed with the particulates, the particulates surround each water droplet and sequester it from the other particulate surrounded water droplets. The resulting composition is in a powder form. However, when compressed, the water will exude from the particulate coating and liquefy on contact with the application surface. Powdered water was discovered in the 1960's and has been used in cosmetics.

U.S. Pat. No. 3,393,155 teaches compositions that are predominantly aqueous based but exist in a dry powder form and methods for their preparation.

U.S. Patent Publication No. 2004/0028719A1, teaches another method for preparing dry water, which involves agitating water and hydrophobic particulates together to form a powdered water composition. The patentee teaches that other ingredients may be present such as water soluble polymers in the aqueous phase.

U.S. Pat. No. 6,290,941 teaches liquid to powder cosmetic compositions in such a form. Patentee teaches that such powder to liquid compositions contain hydrophobically coated silica particles into which are incorporated water and a water soluble polymer. However, one problem with such compositions is that the water tends to seep out of the particles on storage. Water leakage causes the composition to become paste-like with time. These problems are believed to be due to the fact that the water absorbent polymer present in the compositions is water soluble.

While dry water cosmetic compositions are known, they are generally not formulated to exhibit a color change when applied to keratinous surfaces. Such compositions typically exhibit a whitish/beige color in the powder state, and when applied to skin provide essentially the same color. For a variety of reasons, cosmetics that provide color change upon application are desirable commercial products. Such cosmetics give the appearance of being “smart”, that is, that they change color upon application to provide a color that compliments the user's coloring. It has been discovered that the dry powder form is an excellent vehicle for providing cosmetic compositions that form a first resting color to a second application color.

It is an object of the invention to provide powdered water color cosmetic compositions that have a first resting color and a second application color, where the first and second colors are visually distinct.

It is a further object of the invention to provide color cosmetic compositions in the powdered water form that are suitable for use as lipsticks, eye shadows, foundations, mascaras, eye liners, or powders, where such cosmetics have a first resting color and a second application color, and where the first and second colors are visually distinct.

It is a further object of the invention to provide color cosmetic compositions in the powdered water form that may be used to deliver active ingredients to the skin, lips, or lashes, and where the cosmetic changes color upon application.

### SUMMARY OF THE INVENTION

The invention is directed to a powdered water cosmetic composition comprising activatable pigments, a water phase comprised of water droplets, and a particulate phase comprised of non-wetting particulates operable to surround and sequester the individual water droplets from each other in the composition, wherein said activatable pigments prior to activation provide a composition having a first resting color and, upon activation by application to a keratinous surface, a second application color which is visually distinct from the first color.

The invention is also directed to a method for coloring keratinous surfaces by treating with a powdered water cosmetic composition comprising activatable pigments, a water phase comprised of water droplets, and a particulate phase comprised of non-wetting particulates operable to surround and sequester the individual water droplets from each other in the composition, wherein said activatable pigments prior to activation provide a composition having a first resting color and, upon activation by application to the keratinous surface, a second application color.

### DETAILED DESCRIPTION

I. Definitions

A. Activatable, with respect to the pigments, means that they are capable of being activated to develop color. The activation, or color development, may be attributable to the mechanical agitation involved in the process of applying the composition to the desired keratinous surface, the temperature or pH difference found between the composition in the container and as applied to the desired keratinous surface, or upon exposure to water or fluids on the skin or hair. In general, the activatable pigments used in the compositions of the invention will not develop their full color unless and until applied to the keratinous surface. Prior to activation the pigments cause the composition into which they are incorporated to exhibit a first color. After activation, the pigments cause the composition into which they are incorporated to exhibit a second color. The first and second colors are visually distinct, as further described below.

B. First resting color means, with respect to the composition, that when it is in the resting state, e.g. stored in a container, it exhibits one visually distinct color. The activatable pigments present in the composition, prior to activation, will result in a composition that has first color.

C. Non-Wetting, with respect to the particulates, means that they are not wet by, or solvated with, water.

D. Percentages. All percentages mentioned herein are percentages by weight unless otherwise indicated.

E. Powdered Water or Dry Water. Refers to compositions that contain water but where the coalescence of the individual water droplets to form liquid water is prevented because the individual water droplets are surrounded with non-wetting hydrophobic particulates in an amount sufficient to sequester the water droplets from each other while the composition is in the resting state.

F. Second application color means, with respect to the composition, that when it is applied to the keratinous surface (e.g. removed from the resting state), it exhibits a second color that is visually distinct from the first color. The second color is achieved in part due to activation of the activatable pigments so that they develop color upon application to the desired keratinous surface.

G. Visually distinct means, with respect to color differences, that they can be observed with the naked eye.

H. Wettability means the ability of a solid surface (in this case the non-wetting particulate) to be wet by the water droplets comprising the water phase of the composition. The degree of wetting is generally determined by measurement of the contact angle between the solid and liquid phases when both are at thermal equilibrium.

II. The Composition

The powdered water compositions of the invention are cosmetic compositions comprising the following ingredients.

A. Water

The powdered water compositions of the invention generally contain from about 1-85%, preferably from about 2-60%, more preferably from about 5-55% by weight water. In the compositions of the invention, the individual water droplets present may have particle sizes ranging from about 5 to 200 microns, preferably from about 8 to 100, more preferably from about 10 to 80 microns.

B. Non-Wetting Particulates

The powdered water compositions of the invention comprise from about 0.1-85%, preferably from about 0.5-80%, more preferably from about 1-75% by weight of the total composition of non-wetting particulates, preferably silica. Such particulates may have particle sizes ranging from about 0.005 to 75 microns.

Suitable non-wetting particulates are silicas or fumed silicas that tend to be hydrophobic in nature. In general, such silicas are made by vapor phase hydrolysis of silicon tetrachloride vapor in a hydrogen or oxygen flame. The resulting combustion process creates silicon dioxide molecules which condense to form particles, which in turn collide, attach, and sinter together to form a three dimensional network. In many cases the resulting silica has considerable hydroxyl functionality, meaning that it will be hydrophilic in nature. In order to make the silica hydrophobic for certain desired applications, the silica may be substituted with C₁₋₆ alkyl or alkoxy groups, in particular methyl, ethyl, or propyl groups. Suitable hydrophobic silicas include those that are surface treated or bonded with silicones such as dimethicone or methicone, or where the silica is prepared in such a manner that it has an alkyl or alkoxy functionality sufficient to provide the desired hydrophobic character.

Examples of suitable hydrophobic silicas are sold by DeGussa under the Aerosil® brand, more specifically Aerosil® R, which indicates hydrophobic character. Included within the Aerosil® R silicas suitable for use in the compositions of the invention are Aerosil® R 202, 972, 974, 805, 812S, and so on. In one preferred embodiment, Aerosil® R202, a hydrophobic fumed silica after treated with polydimethylsiloxane may be used. Suitable hydrophobic silicas are also sold by Cabot Corporation under the Cab-O—Sil® brand such as Cab-O—Sil® TS-530 which is silica treated with hexamethyldisilazane, Cab-O—Sil® TS-720 which is silica treated with dimethicone, and so on.

The non-wetting particulates are capable of surrounding a water droplet to form a coating thereon which is sufficiently strong to prevent the water droplets present in the composition from coalescing to form liquid water.

C. Activatable Pigments

The powdered water cosmetic composition of the invention also contains activatable pigments. Pigments are generally made activatable by coating with a particular material that causes them to be soluble or dispersible in water, or in the particulate phase of the dry water composition. While it is preferred that the activatable pigments be suspended in the particulate phase of the dry water composition, such pigments may be dispersed in the water phase or the oil phase, if any, of the composition.

Suitable pigments that may be treated to become activatable include inorganic or organic pigments water soluble or water insoluble. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments also generally consist of insoluble metallic salts of certified color additives, referred to as the Lakes.

Also suitable are inorganic pigments such as iron oxides, including red, black, yellow and the like; ultramarines, chromium, chromium hydroxide colors, and mixtures thereof.

Such may be regular sized, ranging from 35-200, from about 40 to 100 microns, or such pigments may be in the microfine particle size form, where the particle sizes range from about 0.05-34 microns, preferably from about 0.1-25 microns, more preferably from about 1-25 microns, most preferably from about 1-15 microns in diameter.

In one preferred embodiment of the invention the pigments are water soluble and become activatable by being treated with a material that causes the pigments to be dispersible in the particulate phase of the composition. Most preferred is where the pigments present in the composition are water soluble pigments treated with an ingredient that causes such pigments to be dispersible in the particulate phase. One preferred embodiment is where such pigments will be dispersible rather than soluble in both water, oil if present, and the particulate phase of the composition. The dispersion of the activatable pigments in the particulate phase of the dry water composition provides a composition that has a first resting color. The first resting color may be a more neutral or lightly tinted color because the full color of the pigments is not visible when suspended or dispersed in the particulate phase, and when such pigments have not been activated. In one embodiment of the invention, the color of the composition in the resting state when the activatable pigments are suspended or dispersed in the particulate phase, the composition may be white, grayish white, whitish blue, or a variety of other colors depending on the color of the powders or other non-activatable pigments that are used in the composition. However, when the composition is applied to skin, the activatable pigments diffuse out of the water droplets and onto the keratinous surface, causing the composition to develop to a different color on the keratinous surface when it is applied. The color development may be attributed to the contact of the pigment with the water or lipids on the skin, the mechanical agitation generally found in the application process, or the temperature or pH change when the composition is applied to the keratinous surface.

In the case of a composition applied to the keratinous surface for the purpose of coloring, such as foundation, blush, lip color, mascara, the development of the color directly on the keratinous surface from a non matching color to a color that matches or compliments the user's skin or hair gives the consumer the impression that the composition is “smart” and capable of changing color to exactly match or compliment her coloration.

If the pigments are coated with a substance in order to make them soluble, or dispersible or suspendable, in either the water phase, oil phase, or particulate phase of the dry water composition, a variety of materials are suitable including polysaccharides, carbohydrates, silicone polymers, organic polymers, or biological polymers. While such treatment materials may, in general, be generally known to be hydrophilic in character, sometimes the treatment will cause the particle to be dispersible rather than soluble in water. Alternatively, the pigments may be lipophilic in character and more readily dispersible in the particulate phase of the composition.

Examples of biological polymers suitable as pigment coating materials for the pigments used in the compositions of the invention include those having repeating monomers that are generally of the carbohydrate type. Included are materials obtained from edible grains such as corn starch; or chemically modified materials obtained from edible grains. Examples of such biological polymers include, but are not limited to, biosaccharide gum; chitosan and derivatives thereof such as butoxy chitosan, carboxymethyl chitosan, carboxybutyl chitosan, chitosan gluconate, chitosan adipate, chitosan glycolate, chitosan lactate, etc; chitin and derivatives thereof such as carboxymethyl chitin, chitin glycolate; cellulose and derivatives thereof such as cellulose acetate; microcrystalline cellulose; distarch phosphate; sodium hyaluronate; soluble proteoglycan; galactoarabinan; glycosaminoglycans; glycogen; sclerotium gum; dextran; starch and derivatives thereof, and the like.

Examples of carbohydrates suitable for use in coating pigments for use in the compositions of the invention may be described as polyhydroxy aldehydes or ketones that conform to the general structure:
Cₓ(H₂O)_{y}
wherein x and y range from about 1 to 1,000,000. The carbohydrates may be monosaccharides, disaccharides, or polysaccharides. Examples of suitable carbohydrates include those set forth on pages 1660 through 1662 of the International Cosmetic Ingredient Dictionary and Handbook, Volume 2, Eighth Edition, 2000, which is published by The Cosmetic, Toiletry, and Fragrance Association (C.T.F.A.), hereby incorporated by reference in its entirety. Such carbohydrates include, but are not limited to, amylodextrin, betaglucan, cyclodextrin, modified corn starch, glycogen, hyaluronic acid, hydroxypropyl cyclodextrin, lactose, maltitol, guanosine, glyceryl starch, triticum vulgare starch, trehalose, sucrose or derivatives thereof; raffinose, sodium chondroitin sulfate, and the like.

Also suitable for treating the pigments are C₁₋₂₀ alkylene glycols or C₁₋₂₀ alkylene glycol ethers either alone or in combination with tri-C₁₋₂₀ alkylsilanes with the alkyl groups ranging from about C₁₋₂₀. Examples of such pigments include PEG alkyl ether alkoxy silane treated pigments including but not limited to PEG-8 methyl ether triethoxysilane treated pigments sold by Kobo under the brand name “SW” pigments.

Also suitable are pigments treated with silicones such as dimethicones having hydrophilic groups also known as dimethicone copolyols or alkyl dimethicone copolyols. In particular, such dimethicones include those having C₁₋₂₀ alkylene oxide repeating units such as ethylene, propylene, and the like. One particular suitable pigment is PEG-12 dimethicone treated pigment sold by Sensient Corporation under the trade name LCW AQ pigments.

Another suitable pigment includes microencapsulated pigments such as iron oxides where the capsule material includes polymeric materials. One particularly suitable example is iron oxide micro-pigments sold by Tagra, Netanya, Israel and distributed by TRI-K Industries under the trade name TAGRA. One type of Tagra cap includes microencapsulated yellow iron oxide, titanium dioxide, boron nitride, ammonio methacrylate copolymer, and triethyl citrate, sold as YellowCap 1.

Particularly preferred is where the composition contains activatable water soluble iron oxide pigments, which are coated or treated with galactoarabinan, which is a polysaccharide obtained from the extraction of the larch tree Larix and such pigments are suspending in the particle phase of the dry water composition. Such pigments are available from Color Techniques under the GA series. For example, a suitable red iron oxide is GA-7293 hydrophilic red iron oxide sold by Color Techniques. Preferred is where from about 10 to 100, preferably from about 20 to 100 parts, more preferably from about 40 to 100 parts by weight of the total pigment present in the composition comprises activatable pigments

C. Other Ingredients

The compositions of the invention may contain other ingredients that enhance the beneficial and aesthetic properties thereof.

1. Water Insoluble Super Absorbent Polymer

The composition of the invention may contain at least one super absorbent polymer that may be water soluble or water insoluble. Various water soluble sodium polyacrylates are suitable. Water insoluble polymers include crosslinked polyacrylate, more particularly crosslinked sodium polyacrylate. While sodium polyacrylates are well known in the art, they tend to be uncrosslinked, which makes them highly water soluble. Most suitable are sodium polyacrylates sold under the brand name Aqua-Keep® by Kobo Products, Inc. including Aqua-Keep® J-440, SAGON Type II, J-550, or 10SH-NF. Particularly preferred is sodium polyacrylate sold by Kobo Products under the brand name Aqua-Keep® 10SH-NF, which has an absorption capacity of about 60 grams per gram of material, an absorption rate of about 8 sec., a bulk density of about 0.87 g/ml, with a particle size of under 100 microns.

Preferably, the superabsorbent polymer used in the compositions of the invention is water insoluble and is dispersed in the particulate phase. When sufficient hydrophobic silica particles surround the water droplet such that it is prevented from coalescing, the water insoluble super absorbent polymer that remains in the particulate phase. Should any water leach out of the water droplets surrounded by silica, it will be absorbed by the polymer. This improves the stability of the composition.

2. Pigments and Powders

The composition of the invention may contain pigments, powders or both which are non-activatable. By non-activatable, is meant that the pigment does not change or develop color upon application to the keratinous surface. If present, suggested ranges of non-activatable pigments or powders or both are from about 0.1 to 95%, preferably from about 0.5-85%, more preferably from about 1-70% by weight of the total composition.

(a). Pigments

Suitable pigments include organic or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

(b). Powders

Suitable powders include colored or non-colored (for example white) non-pigmentatious particles. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, polymethylsilsesquioxane, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof.

The above mentioned pigments and powders may be surface treated with lecithin, amino acids, mineral oil, silicone, lauroyl lysine, fluoro compounds, or various other agents either alone or in combination, which coat the powder surface and render the particles more lipophilic in nature. Preferred is where one or more of the pigments and/or powders present are coated with ingredients that confer such lipophilicity. Particularly preferred are fluoro compounds, more specifically fluorinated paraffinic hydrocarbons or alcohols, such as C9-15 fluoroalcohols, and the like.

In the most preferred embodiment of the invention, more than 75%, preferably more than 80% of the pigments, powders, or both are hydrophobic, or coated with sufficient hydrophobic material to confer hydrophobicity.

3. Humectants, Stabilizers

The composition of the invention may comprise one or more humectants or stabilizers. If present, suggested ranges of humectant are from about 0.01-30%, preferably 0.5-25%, more preferably 1-20% by weight of the total composition. In general, such humectants are water soluble and will be found in the water phase droplets in the composition of the invention. Suitable humectants include materials such as glycols, sugars, and the like. Suitable glycols include polyethylene and polypropylene glycols such as PEG 4-240, which are polyethylene glycols having from 4 to 240 repeating ethylene oxide units; as well as C₁₋₆ alkylene glycols such as propylene glycol, butylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆, preferably C₂₋₄ alkylene glycols, most particularly butylene glycol.

Stabilizers may also be added, which are ingredients that stabilize the composition, particularly the water phase. Examples are water soluble salts and urea or urea derivatives. Stabilizers, if present, may be found in the same ranges as humectants.

4. Botanical Extracts

It may be desirable to include one or more botanical extracts in the compositions. If so, suggested ranges are from about 0.0001 to 10%, preferably about 0.0005 to 8%, more preferably about 0.001 to 5% by weight of the total composition. Suitable botanical extracts are preferably water soluble and will be solubilized in the water droplets. Such extracts include those from plants (herbs, roots, flowers, fruits, seeds) such as flowers, fruits, vegetables, and so on, including acacia (dealbata, framesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, and mixtures thereof.

5. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

The powdered water compositions are prepared by combining the silica and the water phase ingredients and mixing well in a blender at high speed. The remaining ingredients are added to the pre-mix and blended at high speed in the blender.

The resulting compositions are in the powdered form. When applied to skin, the non-wetting particulates that surround the water droplets and prevent them from coalescing into liquid water when the composition is in the resting state, will disperse so that the water present is applied to the skin along with the pigments and powders present. The compositions provide a very light, non-greasy, color to the skin. The composition may be in the form of a foundation, powder, eye shadow, lipstick, blush, bronzer, or any other type of color cosmetic product.

The invention will be further described in connection with the following examples which are set forth for the purposes of illustration only.

### EXAMPLE 1

A powdered water composition suitable as a face powder or foundation makeup that changed color upon application was prepared as follows:

| | Ingredient | % by weight | | |
|---|---|---|---|---|
| 1 | Talc, C9-15 fluoroalcohol phosphates | 31.50 | 31.50 | 31.50 |
| 1 | Methyl paraben | 0.20 | 0.20 | 0.20 |
| 1 | Propyl paraben | 0.10 | 0.10 | 0.10 |
| 1 | Polymethylsilsesquioxane | 1.00 | 1.00 | 1.00 |
| 1 | Sodium polyacrylate* | 0.50 | 0.50 | 0.50 |
| 1 | HDI/trimethylol hexyllactone | 5.00 | 5.00 | 5.00 |
| | crosspolymer, silica | | | |
| 1 | Titanium dioxide | 0.50 | 0.50 | 0.50 |
| 2 | Pigment Blend (Color Techniques | 4.50 | — | — |
| | Galactoarabinan treated)** | | | |
| 2 | Pigment Blend (Kobo Products SW | — | 4.50 | — |
| | pigments)*** | | | |
| 2 | Pigment Blend (Sensient/LCW AQ | — | — | 4.50 |
| | pigments)**** | | | |
| 3 | Water | 47.50 | 47.50 | 47.50 |
| 3 | Sodium Chloride | 2.00 | 2.00 | 2.00 |
| 3 | Diazolidinyl urea | 0.20 | 0.20 | 0.20 |
| 3 | Butylene glycol | 2.50 | 2.50 | 2.50 |
| 3 | Glycerin | 2.00 | 2.00 | 2.00 |
| 4 | Silica, dimethicone (Aerosil R202) | 2.50 | 2.50 | 2.50 |

*cross-linked, water insoluble sodium polyacrylate, Aqua-Keep 10SH-NF, Kobo Products Inc.
**a blend of 50 parts galactoarabinan treated yellow iron oxide GA-7311, 30 parts galactoarabinan treated red iron oxide GA-7293, and 20 parts galactoarabinan treated black iron oxide GA-7403, all purchased from Color Techniques.
***a blend of 50 parts yellow iron oxide, 30 parts red iron oxide, and 20 parts black iron oxide, all treated with PEG-8 methyl ether triethoxysilane, purchased from Kobo Products under the brand name SW pigments.
****a blend of 50 parts yellow iron oxide, 30 parts red iron oxide, and 20 parts black iron oxide, all treated with PEG-12 dimethicone, purchased from Sensient under the brand name LCW AQ pigments.

The phase 1 ingredients were combined in a blender and blended on high for 90 seconds at high speed. The blender was stopped, the mixture scraped off the blender walls with a brush, then blended at high speed for an additional 90 seconds. The mixture was checked for uniformity. The phase 2 ingredients were then added to the phase 1 mixture and gently tumbled, avoiding shearing or chopping, until the pigments were dispersed. The phase 3 ingredients were separately combined and mixed until uniform. The phase 4 ingredients were added to phase 3 mixture in a blender and blended on high for 30 seconds. The blender was stopped to scrape the blender walls of any excess water, and the blending continued for an additional 30 to 45 seconds at high speed. Phases 1 and 2 were combined with 3 and 4 and the mixture gently tumbled until uniform. The resulting compositions was an off white powder. When applied to skin it formed a beige colored, skin matching liquid that dried to a powdery finish.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A powdered water cosmetic composition comprising activatable pigments, a water phase comprised of water droplets, and a particulate phase comprised of non-wetting particulates operable to surround and sequester the individual water droplets from each other in the composition, wherein said activatable pigments prior to activation provide a composition having a first resting color and, upon activation by application to a keratinous surface, a second application color which is visually distinct from the first color.

2. The composition of claim 1 wherein the non-wetting particulates are hydrophobic silica.

3. The composition of claim 3 wherein the hydrophobic silica is silica substituted with C1-6 alkyl or alkoxy groups.

4. The composition of claim 2 wherein the hydrophobic silica is silica substituted with methicone.

5. The composition of claim 2 wherein the hydrophobic silica is silica coated with dimethicone.

6. The composition of claim 1 further comprising a superabsorbent polyacrylate polymer.

7. The composition of claim 1 comprising from about 0.1 to 95% activatable pigments, and from 0.1-95% non-activatable pigments, or powders, by weight of the total composition.

8. The composition of claim 7 wherein the non-activatable pigments or powders are coated with a hydrophobic ingredient.

9. The composition of claim 8 wherein the hydrophobic ingredient is silicone, mineral oil, silk, or a fluoroalcohol.

10. The composition of claim 9 wherein the hydrophobic material is C9-15 fluoroalcohol.

11. The composition of claim 1 comprising, by weight of the total composition, from about 0.1-85% non-wetting particulates, from about 1-65% water, and from about 0.1-95% of activatable pigments, and from about 0.1-80% powders.

12. The composition of claim 11 wherein the non-wetting particulates are silica treated with dimethicone.

13. The composition of claim 11 additionally comprising from about 0.1-20% humectant in the water phase.

14. The composition of claim 11 comprising from about 0.1-50% activatable pigment.

15. The composition of claim 14 wherein the pigments are one or more iron oxides.

16. The composition of claim 14 wherein the powders include mica, titanium dioxide, polymethylsilsesquioxane, boron nitride, or mixtures thereof.

17. The composition of claim 1 which is a foundation makeup.

18. The composition of claim 1 which is an eye shadow.

19. The composition of claim 1 which is a powder.

20. The composition of claim 1 which is a lipstick.

21. The composition of claim 1 wherein the activatable pigments are suspended in the particulate phase of the composition.

22. The composition of claim 21 wherein the activatable pigments are water dispersible.

23. The composition of claim 22 wherein the water dispersible activatable pigments comprise iron oxides.

24. The composition of claim 21 wherein the activatable pigments are treated with a mono-, di-, or polysaccharide.

25. The composition of claim 24 wherein the polysaccharide is galactoarabinan.

26. The composition of claim 1 wherein the first resting color is off white or gray.

27. The composition of claim 26 wherein the second application color is beige.

28. The composition of claim 1 wherein the activatable pigment comprises water soluble iron oxides treated with a material that causes them to be dispersible in the particulate phase of the composition, and prior to activation to form the first resting color.

29. The composition of claim 28 wherein the second application color is formed when the composition is applied to the keratinous surface.

30. A method for coloring keratinous surfaces by treating with a powdered water cosmetic composition comprising activatable pigments, a water phase comprised of water droplets, and a particulate phase comprised of non-wetting particulates operable to surround and sequester the individual water droplets from each other in the composition, wherein said activatable pigments prior to activation provide a composition having a first resting color and, upon activation by application to the keratinous surface, a second application color.
